(19) United States
(12) Patent Application Publication
(10) Pub. No.: US 2003/0083496 A1
(43) Pub. Date: May 1, 2003

(54) High-pressure process for the preparation of melamine from urea

(76) Inventor: Hans Vrijenhoef, HE Brielle (NL)

Correspondence Address: ROTHWELL, FIGG, ERNST & MANBECK, P.C., 1425 K STREET, N.W., SUITE 800, WASHINGTON, DC, 20005, US

(21) Appl. No.: 10/303,787

(22) Filed: Nov. 26, 2002

Related U.S. Application Data
(63) Continuation of application No. 09/869,271, filed on Jun. 26, 2001, now abandoned.
Application No. 09/869,271 is a 371 of international application No. PCT/FI99/01090, filed on Dec. 29, 1999.

(30) Foreign Application Priority Data
Dec. 31, 1998 (FI) 982847

(51) Int. Cl. C07D 251/60

(52) U.S. Cl. 544/201

(57) ABSTRACT

The invention relates to a process for the preparation of melamine from urea comprising the steps of:

a) introducing urea melt and hot ammonia gas into a melamine reactor having a pressure within the range 50-150 bar and a temperature within the range 360-470° C., to obtain a melamine melt and off-gases;

b) separating said off-gases from said melamine melt;

c) introducing said melamine melt and ammonia gas into an evaporator having a pressure within the range 1-15 bar and a temperature within the range 290-520° C., to obtain a melamine-containing gas mixture; and

d) introducing said melamine-containing gas mixture into a cooler having a pressure within the range 1-15 bar and a temperature within the range 0-250° C., to convert the gaseous melamine to solid high purity melamine.

The present invention relates to a high-pressure process for the preparation of highly pure melamine from urea.

It is known that melamine can be prepared from urea at a temperature of 390-410° C. according to the following reaction formula:

6H₂N—CO—NH₂→C₃N₃(NH₂)₃+6NH₃+3CO₂

The reaction is strongly endothermic. The heat requirement is 649 kJ per melamine mole, when the heating af the urea from 135° C. (melting point of urea) to the reaction temperature is included.

Users require a very high purity of melamine; 99.8% and 99.9% are typical degrees of purity in product specifications. For this reason its production processes often include a complicated purification section involving a large quantity of apparatus.

There are two basic types of melamine production processes using urea as the raw material, namely, catalytic low-pressure processes and high-pressure processes in which no catalyst is used. In the former, the reactor pressure is approx. 10 bar or lower, in the latter higher than 80 bar (Ullmann's Encyclopedia of Industrial Chemistry, 5th edition, Vol. A 16, p. 174).

In typical low-pressure processes, a fluid-bed reactor is used in which the catalyst is fluidized with gaseous ammonia or a mixture of ammonia and carbon dioxide. The melamine emerges in gaseous state from the reactor. The fact that corrosion is less than in high-pressure processes is regarded as one of the advantages of low-pressure processes. The best known users of low-pressure processes are BASF (Hydrocarbon Processing, September 1969, p. 184), Chemie Linz (Hydrocarbon Processing, November 1966, p. 146), and DSM/Stamicarbon (Chem. Eng., May 20, 1968, p. 124), each of which has developed its own process version.

In typical high-pressure processes the reaction takes place in a liquid phase. In this case the reactor is full of molten melamine mixed to some degree with molten raw material, i.e. urea, and intermediate reaction products. Also present in the mixture there are gas bubbles consisting of ammonia and carbon dioxide and a small amount of gaseous melamine. The required high amount of reaction heat is usually generated by intra-reactor heating elements, in which the heat is generated by means of electricity or, for example, a circulating hot salt melt.

Smaller apparatus size can be deemed to be one of the advantages of high-pressure processes over low-pressure processes. A reaction taking place in a liquid phase clearly requires less space. Furthermore, the process apparatus in which gas is treated remain moderate-sized owing to the high pressure. Another advantage is the high pressure of the obtained product gas, a mixture of ammonia and carbon dioxide. This gas is often used for the preparation of urea and, being pressurized, it is better suited for this purpose as such.

The Montedison process (Ausind) is a typical high-pressure melanine production process (Ullmann's Encyclopedia of Industrial Chemistry, 5th edition, Vol. A 16, p. 177). As in other melamine processes, urea melt and hot ammonia are introduced into a reactor. The reactor conditions are 70 bar and 370° C. From the reactor the mixture of melamine melt and product gases is directed to a quencher, into which water containing ammonia and carbon oxide is also introduced. The temperature of the quencher is 160° C. and its pressure 25 bar. From this quencher the reactor off-gases are fed for further use, for example for the production of urea or fertilizers. The melamine is recovered from the slurry by a highly multiple-stage further treatment, which includes the removal of ammonia and carbon dioxide, the dissolving of the melamine in a large amount alkaline water, removal of color with activated carbon, crystallization, filtration, drying, and packaging.

The Montedison process has two significant disadvantages. First, the product gas is obtained at a relatively low pressure, only 25 bar. Second, the number of process stages is very high, since the impure product obtained from the reactor requires a thorough purification treatment. Furthermore, the purification apparatus is relatively large in size, since the pressures in it are already low.

In the Nissan high-pressure process (Chemical Economy & Engineering Review, Vol. 8, (1976), No. 1, 2, p. 35), advantages have been gained over the Montedison process at least with respect to the off-gases, as is evident from the following. Also in the Nissan process, melamine melt and hot ammonia are fed into the reactor. The temperature and the pressure are 400° C. and 100 bar. In the upper section of the reactor, the melamine melt and the gases are separated. The gases are directed into a scrubbing tower, in which they are scrubbed with urea melt. The melamine present in the gas dissolves in the urea melt. At the same time the gases cool to approx. 200° C. The product gas is thus obtained at a pressure of 100 bar and in anhydrous state, which may be a considerable advantage in terms of its further use. The urea melt to be used as raw material is fed in via the scrubbing tower. There it heats up and water is removed from it (reacts with urea). The melamine melt is dissolved in an aqueous ammonia solution. This solution is maintained under ammoniacal pressure at 180° C. for a certain period, during which the impurities are said to be eliminated. Thereafter follows a further treatment with numerous apparatus, including filtration and crystallization. Thus the number of unit operations and apparatus is really high, which raises the process costs.

Melamine Chemicals has developed a process (U.S. Pat. No. 4,565,867) in which the quantity of apparatus is quite small as compared with the Montedison and Nissan processes. The reactor, into which urea melt and hot ammonia are fed, operates at a temperature of 370-425° C. and under a pressure of 110-150 bar. The mixture of melamine melt and product gases is fed into a gas separation tank, in which the product gases are separated. The gas separation tank operates under the same conditions as the reactor. After a scrub with urea, the product gases are directed for further use. As in the Nissan process, the product gases are obtained at a pressure of approximately 100 bar and in anhydrous state. As in the Nissan process, the urea melt is fed into the reactor via the urea scrubber. From the gas-separation tank the melamine melt is directed to a quencher unit, in which it is cooled rapidly by means of, for example, liquid ammonia or water. Crystalline melamine is obtained, which is withdrawn via the bottom of the quencher unit and taken to drying. There are no actual purification stages. The quantity of apparatus in the process is really small as compared with the processes described above. However, the purity of the product is only 96-99.5%. In this respect the process is not competitive with the processes described above. Achieving a competitive purity by the Melamine Chemicals process would require that a purification process, for example one similar to those in any of the above processes, be installed after the process. Thereby the advantages of the process, i.e. small quantity of apparatus and consequent cost efficiency, would be eliminated.

Purification of the product by vaporization has been propose even previously. One of the earliest melamine preparation patents (GB Patent 800 722) includes an example in which approx. 9 kg of ammonia per one kilogram of melamine product is fed into a reactor which operates at the temperature of 400° C. and under a pressure of 40-80 bar. The amount of ammonia is in this case so high that all of the produced melamine is vaporized into the gas phase. The promoting effect of ammonia on melamine vaporization is based on the fact that it reduces the partial pressure of melamine in the gas phase. When the reaction equation presented in the foregoing and the additional ammonia are taken into account, a stoichiometric calculation shows that the composition of the gas leaving the reactor will be, in percent by volume: ammonia 94.8%, carbon dioxide 3.9%, and melamine 1.3%. Thus the melamine has to be recovered from a very large amount of gas. Furthermore, before the large amount of ammonia can be recycled into the reactor for reuse, carbon dioxide has to be separated from it. Quite pure melamine could probably be produced by a process such as this. Indeed, the said patent states that the only impurity present in the product was unreacted urea. However, the process is uneconomical owing to the large gas amounts and the related separation operations, which are the separation of the melamine from a very large amount of gas and the separation of the carbon dioxide from a very large amount of ammonia.

In the process described above, the ammonia amount required could be decreased by lowering the reactor pressure or by raising the temperature. In this case the melamine would vaporize more easily into the gas phase, and ammonia would not be needed in such a large amount to lower its partial pressure. If the reactor pressure is lowered to below 50 bar, there will form in the reactor a solid which will complicate and ultimately hinder the operation of the reactor. This has been noted in, for example, patent U.S. Pat. No. 3,484,440, column 1, line 65. Raising the temperature to above 400° C. for its part increases corrosion and weakens the structural materials of the reactor.

Nissan has investigated a process (U.S. Pat. No. 3,484,440) which resembles that described above but can be managed with a smaller amount of ammonia, in which case the melamine content is many times higher in the off-gas. In this process, hot ammonia is fed into the reactor at a rate of 0.2-1.0 g per each gram of urea. The reactor conditions are 360-400° C. and 50-150 bar. From the reactor the liquid melamine melt and in its midst the product gases (ammonia and carbon dioxide) are directed via a heater into a vaporizer. A pressure of 40-100 bar and a temperature of 420-480° C. are maintained in the vaporizer. When the pressure and the temperature are selected suitably within these limits, all of the melamine can be caused to vaporize into the gas phase. For example, when ammonia is fed into the reactor at a rate of 0.2 g per one gram of urea, the concentration of melamine in the leaving gas phase will be approx. 7% by volume, calculated stoichiometrically. When the feed of ammonia is 1 g per one gram of urea, the melamine content in the gas will be approx. 3.2% by volume. For example, the first-mentioned case (melamine 7% by volume) requires a pressure of approx. 72 bar and a temperature of 480° C. or, for example, a pressure of 40 bar and a temperature of 455° C. These pressure and temperature values were taken from a diagram in the patent. Before vaporization, however, the melamine is allowed to remain in the vaporizer in the form of a liquid melt for at minimum one hour. Thus the impurities formed in the reaction are caused to reconvert into melamine. From the vaporizer the melamine is directed to a separator, in which it is cooled with water, and will crystallize, the temperature being 150° C. and the pressure approximately atmospheric. In the patent text it is stated that the purity of the product thus obtained is 99%, and the purity obtained in the example in the patent is 99.2%. A product such as this is not competitive in terms of purity. Users require a 99.9% purity of melamine so commonly that this requirement is mentioned in a well-known handbook in the field (Ullmann's Encyclopedia of Industrial Chemistry, 5th edition, Vol. A 16, p. 179). Achieving this purity by the process last described would require a separate purification process, for example dissolving and recrystallization, as do most melamine processes. This would be an expensive addition and would make the entire sense of the vaporization questionable; indeed, purification could be started directly on the product obtained in molten state from the reactor, without wasting energy on vaporization.

The prior-art melamine production processes can be divided into two categories. In most low-pressure processes and the Nissan (meaning the first-mentioned of the two Nissan processes referred to) and Montedison high-pressure processes, evidently a competitive purity is achieved, but the processes involve a multiple-stage purification section with a large quantity of apparatus, which clearly increases both the operating costs and the investment. The processes described in patents U.S. Pat. Nos. 4,565,867 and U.S. Pat. No. 3,484,440 constitute the second category. They are in the same category in terms of the quantity of apparatus, and they do not have the multiple-stage purification section mentioned above. However, the purity of the product obtained from them is not competitive.

The Applicants have previously developed a high-pressure process for the preparation of melamine wherein the number of apparatus is substantially the same as in the above Melamine Chemicals process (U.S. Pat. No. 4,565,867), but the purity of the product is in the order of 99.9% or above. This process is based i.a. on evaporating the melamine melt obtained from the melamine reactor and subsequently crystallizing the melamine from the gas phase. More specifically, this previous process disclosed in WO 95/01345 comprises the steps of:

i) feeding molten urea and hot ammonia gas into a reactor having a pressure within the range 50-150 bar and a temperature within the range 360-430° C., to obtain a reaction product containing a liquid melamine melt and a gas mixture;

ii) separating the gas mixture from the liquid melamine melt;

iii) evaporating the liquid melamine melt thus obtained in an evaporator; and

iv) cooling the melamine-containing gas obtained from the evaporator, whereby the melamine is crystallized in a very pure state.

According to WO 95/01345 the melamine melt obtained from the melamine reactor can be evaporated by lowering the pressure or by raising the temperature or by feeding ammonia gas into the evaporator or by using two or all of these three methods. Specifically disclosed pressures are within the range 20-90 bar and temperatures are within the range 420-470° C. In the specification it is stated that at a pressure of 50 bar and a temperature of 450° C. at minimum approx. 2.4 kg of ammonia per one kilogram of melamine is needed in the evaporator, meaning that the molar ratio of ammonia to melamine is at least 17.6:1.

Furthermore, according to a preferred embodiment of WO 95/01345 the cooling of the melanine-containing gas is carried out by contacting said gas directly with liquid ammonia in a cooler at a temperature below 130° C. and at a pressure below 40 bar.

The aim of the present invention is to further improve the process disclosed in WO 95/01345 especially in respect of simplicity of performance and energy consumption or heat recovery in order to make the process more economic.

Thus, the present invention provides a process for the preparation of melamine from urea comprising the steps of:

a) introducing urea melt and hot ammonia gas into a melamine reactor having a pressure within the range 50-150 bar and a temperature within the range 360-470° C., to obtain a melamine melt and off-gases;

b) separating said off-gases from said melamine melt;

c) introducing said melamine melt and ammonia gas into an evaporator having a pressure within the range 1-15 bar and a temperature within the range 290-520° C., to obtain a melamine-containing gas mixture; and

d) introducing said melamine-containing gas mixture into a cooler having a pressure within the range 1-15 bar and a temperature within the range 0-250° C., to convert the gaseous melamine to solid high purity melamine.

Steps a) and b) are preferably carried out as explained in WO 95/01345.

In step c) the pressure is within the range 1-15 bar, preferably within the range 5-15 bar, and the temperature is within the range 290-520° C., preferably within the range 310-520° C. and more preferably within the range 360-480° C. At a pressure of 2 bar the temperature is preferably within the range 300-350° C. and at a pressure of 10 bar the temperature is preferably within the range 340-425° C. In the evaporator the molar ratio of ammonia to melamine is preferably between 1:1 and 17:1, more preferably between 7:1 and 17:1. Preferably superheated ammonia gas is introduced into the evaporator. The temperature of superheated ammonia is typically about 540° C. The melamine retention time in the evaporator is preferably less than half an hour, and especially preferably less than 10 minutes.

Preferably following conditions are prevailing in the evaporator:

the pressure is within the range 5-15 bar,

the temperature is within the range 360° C.-480° C., and

the molar ratio of ammonia to melamine is 7:1 to 17:1.

In the evaporator the temperature, pressure and molar ratio of ammonia to melamine are dependent on each other. For example at a pressure of 10 bar and a molar ratio of 7:1, the temperature required to evaporate all melamine is about 440° C., and at a pressure of 10 bar and a molar ratio of 17:1, the temperature required to evaporate all melamine is about 400° C.

In cooling step d) the pressure is within the range 1-15 bar, preferably within the range 5-15 bar, and the temperature is within the range 0-250° C., preferably within the range 30-230° C. Preferably the pressure is the same as in the evaporator. A cooling medium is preferably introduced into the cooler and contacted directly with the melamine-containing gas.

In a first embodiment said cooling medium is liquid ammonia wherein the cooling is effected by evaporating ammonia. There is no liquid ammonia present in the cooler, and cooler and crystal separation can be combined into one apparatus for example of cyclone-type. Evaporating ammonia and ammonia gas originating from the evaporator are withdrawn from the cooler and at least a portion thereof is condensed to form liquid ammonia to be recycled into the cooler as cooling medium. The heat recovered from the ammonia cooling can be utilized for heating ammonia e.g. fresh liquid ammonia, to be introduced into the evaporator.

In a second embodiment of the invention said cooling medium is gaseous ammonia. As in the first embodiment there is no liquid ammonia present in the cooler, and cooler and crystal separation can be combined into one apparatus for example of cyclone-type. Gaseous ammonia whereof a part originates from the cooling medium and a part from the evaporator is withdrawn from the cooler, and at least a portion thereof is cooled to form gaseous ammonia to be recycled into the cooler as cooling medium. The heat recovered from the ammonia cooling can be utilized for heating ammonia e.g. fresh liquid ammonia, to be introduced into the melamine reactor.

According to the invention the off-gases separated from the melamine melt can be introduced into an absorption device for recovering the small amount of melamine present in the off-gases. Preferably the off-gas treatment is carried out by absorbing melamine from the off-gases in a counter-current direct contact system wherein urea is the absorbent. Typically the absorption device comprises an absorption column. In order to lower the urea loss in the off-gas of the absorption column a condenser is provided for condensation of urea-absorbent. This apparatus recovers most of the urea present in the off-gas of the absorption column. A one or two stage absorber can be used. However, to ensure that all off-gases are cooled sufficiently for recovery of melamine, the two stage absorber is preferred. The heat recovered from the urea condenser can be used for heating ammonia to be introduced into the melamine reactor. Alternatively the heat recovered from the urea condenser can be used for steam production. The cooled off-gases containing mainly ammonia and carbon dioxide can be recycled to a urea plant. The absorption device preferably operates at essentially the same pressure as the melamine reactor. The operating temperature of the absorption device has to be above 180° C. to avoid carbamate formation and below 260° C. in order to suppress the formation biureth and triureth. The temperature is preferably within the range 230-240° C.

The invention will be described below in greater detail with reference to the accompanying drawings, in which

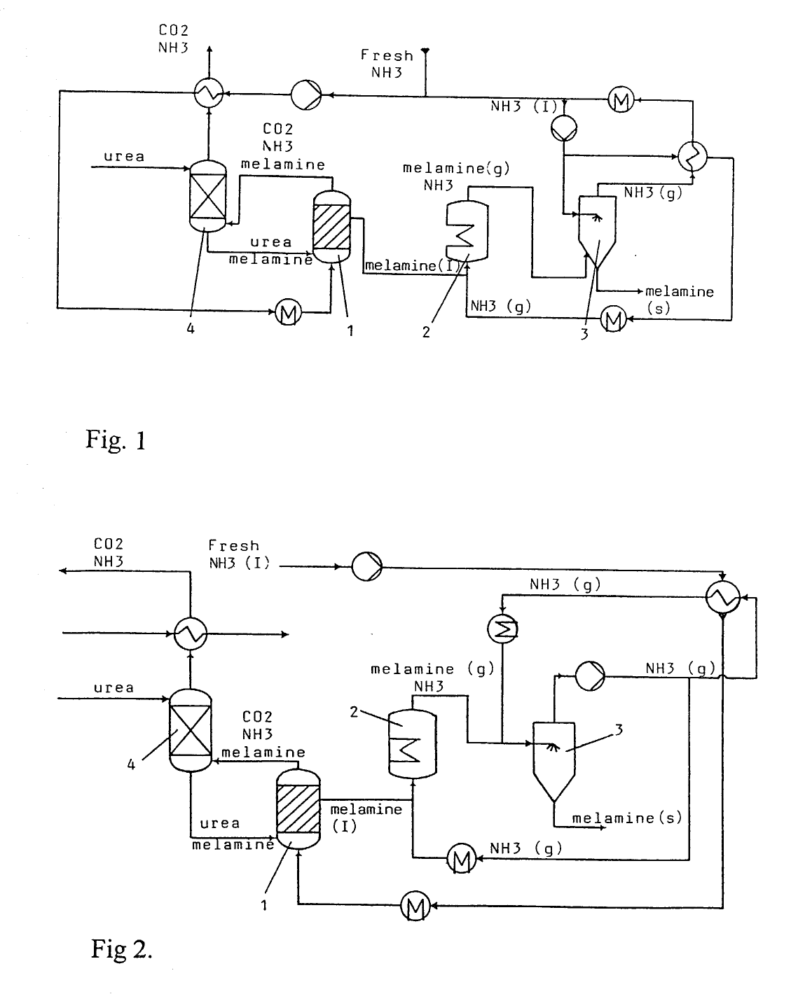

FIG. 1 illustrates a process diagram of a system which can be used for carrying out a first embodiment of the process of the present invention, and

FIG. 2 illustrates an other process diagram of a system which can be used for carrying out a second embodiment of the process of the present invention.

Both process diagrams of FIG. 1 and FIG. 2 comprise from left to right four sections, namely Off-gas treatment—Melamine reactor—Evaporator—Cooler.

In accordance with FIG. 1, urea melt containing small amounts of absorbed melamine from the absorption device 4 and ammonia, preferably hot ammonia are fed into the melamine reactor 1 via different pipelines. The conditions prevailing in the reactor are those of a typical high-pressure process, i.e. the temperature is 360-470° C. and the pressure within the range 50-150 bar. Preferably the temperature is about 400° C. and the pressure about 100 bar. The heating is effected by internal heating elements. The reactor off-gases typically containing up to about 1% melamine, the balance being ammonia, carbon dioxide and unreacted urea, are separated from the melamine melt in the upper section of reactor 1 and directed to the absorption device 4 wherein melamine is recovered from the off-gases by using molten urea introduced from a urea plant as the absorbent.

In absorption device 4 melamine is absorbed from the off-gases by using a counter-current direct contact system followed by condensation of the urea absorbent in a separate condenser. A one or two stage absorber can be used. However, to make sure that all off-gases are cooled sufficiently for recovery of melamine the two stage absorber is preferred. The main purpose of the off-gas treatment is recovery of melamine. Other purposes are preheating the urea melt, cooling the off-gases and dehydration of urea. The advantages of the above described off-gas treatment as compared to previously known systems are that the present system is simple and does not need any pumps or mixers, has a lower residence time (typically only few minutes), results in better dehydration of urea and higher preheat of urea, and needs lower heat input in the melanine reactor. The operating pressure of the absorption device is preferably the same as for the melamine reactor, i.e. within the range 50-150 bar, preferably about 100 bar. The operating temperature of the absorption device is within the range 180-260° C., preferably within the range 230-240° C. The heat recovered from the urea condenser of the off-gas treatment can be used for evaporating fresh liquid ammonia. As shown in FIG. 1 the evaporated ammonia is further subjected to hot salt superheating to obtain hot ammonia to be fed into reactor 1.

The melamine melt obtained in reactor 1 is directed to the evaporator 2. In the evaporator the temperature is within the range 290-520° C. and can for example be about 400° C., and the pressure is within the range 1-15 bar, preferably within the range 5-15 bar. A preferred pressure for the evaporator of the system shown in FIG. 1 is about 10 bar. The evaporator can be heated by, for example, internal heating elements. Ammonia gas is fed into the evaporator in order to evaporate the melamine. The ammonia gas fed into the evaporator can be superheated ammonia gas having a temperature of about 540° C. The molar ratio of ammonia to melamine in the evaporator is preferably between 1:1 and 17:1, more preferably between 7:1 and 17:1. As compared to the Applicants previous WO 95/01345 the evaporator of the present invention operates at a substantially lower pressure, and with a lower molar ammonia/melamine ratio.

The gas mixture containing melamine and ammonia is directed from the evaporator 2 to the cooler 3. Simultaneously a cooling medium is introduced into the cooler and contacted directly with the gas mixture. In the embodiment shown in FIG. 1 the cooling medium is liquid ammonia. The liquid ammonia, when evaporating, will bind the heat released in the cooling and in the crystallization of the melamine. Ammonia gas introduced into the cooler together with the melamine and evaporated cooling medium ammonia discharge from the upper section of the cooler. The pressure of the cooler is within the range 1-15 bar, preferably within the range 5-15 bar, and the temperature is within the range 0-250° C., preferably within the range 30-230° C. A preferred pressure for the cooler of the system shown in FIG. 1 is about 10 bar. In this evaporative ammonia cooling no liquid ammonia is present, and the cooler and crystal separation can be combined into one apparatus for example of cyclone-type. A portion of the gaseous ammonia withdrawn from the cooler is condensed to form liquid ammonia for reuse as cooling medium in the cooler. Heat recovered from the ammonia cooling can be used for heating ammonia to be introduced into the evaporator.

The melamine will be recovered in crystalline form having the same high purity as the melamine produced according to the Applicants WO 95/01345.

FIG. 2 shows a second embodiment of the process of the present invention. In order to avoid repetition only those features that differ from those described above will be discussed. As to the off-gas treatment section, the heat recovered from the urea condenser is used for steam production. In evaporator 2 the operating pressure is about 10 bar. In cooler 3 the operating pressure is about 10 bar. Furthermore, in the embodiment shown in FIG. 2 the cooling medium is gaseous ammonia. A portion of the gaseous ammonia withdrawn from the cooler is cooled to form gaseous ammonia for reuse as cooling medium in the cooler. The heat recovered from the ammonia cooling can be utilized for preheating fresh liquid ammonia which subsequently is subjected to superheating, for example hot salt superheating, and then fed into the melamine reactor 1. The remainder of the gaseous ammonia withdrawn from the cooler is subjected to superheating, for example hot salt superheating, and fed into evaporator 2.

### EXAMPLE

For testing the operating limits of the evaporation phase of the melamine process, melamine (>91 wt-%) containing melam (concentration below the detection limit 100 ppm), melem 8.0 wt-%, ammeline 0.03 wt-% and ureidomelamine 0.14 wt-% as impurities determined by high pressure liquid chromatography (HPLC) was introduced into the evaporator together with ammonia gas. The molar ratio of ammonia to melamine in the feed mixture to evaporator was 15:1 and the total pressure was 10 bar, thus the temperature needed to evaporate melamine totally was about 400° C. (406° C., measured). The residence time of melamine in the evaporator was one hour to make sure that all melamine was totally evaporated and to allow certainly enough time for the purifying reactions to take place. In the evaporator the impurities of melamine, like melem were converted into melamine resulting in purification of melamine which evidently showed that the used amount of excess ammonia and the used pressure were sufficient. After evaporation the gas mixture was cooled rapidly below 100° C. by contacting it with liquid ammonia. The resulting solid crystalline melamine was analysed for impurities.

The concentrations of impurities after the evaporation were melam (concentration below the detection limit 100 ppm), melem 0.02 wt-%, ammeline 0.04 wt-% and ureidomelamine 0.01 wt-% resulting in>99.9 wt-% melamine.

These results show that when melamine which contains impurities before evaporation is evaporated according to the method described by this invention, purifying phenomena take place at the pressures, temperatures and molar ratios of NH3/Melamine used in the feed mixture. The resulting melamine is very pure (>99.9%).

This example proofs that very pure melamine can be produced economically by evaporation at low pressures and temperatures which decrease the cost of equipment. Furthermore, because the required amount of ammonia in the feed mixture is low, the required amount of ammonia to be evaporated and circulated is low thus decreasing the operational costs due to lowered energy consumption in ammonia evaporation and pumping.

1. A process for the preparation of melamine from urea comprising the steps of:
a) introducing urea melt and hot ammonia gas into a melamine reactor having a pressure within the range 50-150 bar and a temperature within the range 360-470° C., to obtain a melamine melt and off-gases;
b) separating said off-gases from said melamine melt;
c) introducing said melamine melt and ammonia gas into an evaporator having a pressure within the range 1-15 bar and a temperature within the range 290-520° C., to obtain a melamine-containing gas mixture; and
d) introducing said melamine-containing gas mixture into a cooler having a pressure within the range 1-15 bar and a temperature within the range 0-250° C., to convert the gaseous melamine to solid high purity melamine.

2. The process according to claim 1 wherein the pressure of the evaporator is within the range 5-15 bar.

3. The process according to claim 2 wherein the pressure of the evaporator is within the range 8-12 bar.

4. The process according to any of claims 1 to 3 wherein the temperature of the evaporator is within the range 300-520° C.

5. The process according to any of claims 1 to 4 wherein the molar ratio of ammonia to melamine in the evaporator is 1:1 to 17:1.

6. The process according to claim 5 wherein said molar ratio is 7:1 to 17:1.

7. The process according to any of claims 1 to 6 wherein following conditions are prevailing in the evaporator:
the pressure is within the range 5-15 bar,
the temperature is within the range 360° C.-480° C., and
the molar ratio of ammonia to melamine is 7:1 to 17:1.

8. The process according to any of claims 1 to 7 wherein superheated ammonia is introduced into the evaporator.

9. The process according to any of claims 1 to 8 wherein a cooling medium is introduced into the cooler and contacted directly with the melamine-containing gas.

10. The process according to claim 9 wherein said cooling medium is liquid ammonia.

11. The process according to claim 10 wherein gaseous ammonia is withdrawn from the cooler and at least a portion thereof is condensed to form liquid ammonia to be reused as cooling medium in the cooler.

12. The process according to claim 11 wherein heat recovered from the ammonia cooling is used for heating ammonia to be introduced into said evaporator.

13. The process according to claim 9 wherein said cooling medium is gaseous ammonia.

14. The process according to claim 13 wherein gaseous ammonia is withdrawn from the cooler and at least a portion thereof is cooled to form gaseous ammonia to be reused as cooling medium in the cooler.

15. The process according to claim 14 wherein heat recovered from the ammonia cooling is used for heating ammonia to be introduced into said melamine reactor.

16. The process according to any of the preceding claims wherein said off-gases separated from the melamine melt are introduced into an absorption device for recovering melamine present in the off-gases.

17. The process according to claim 16 wherein melamine is absorbed from the off-gases in a counter-current direct contacting system with urea as absorbent followed by condensation of the absorbent in a condenser.

18. The process according to claim 17 wherein heat recovered from the urea condenser is used for heating ammonia to be introduced into said melamine reactor.

19. The process according to any of claims 16 to 18 wherein the absorption device operates essentially at the same pressure as the melamine reactor and at a temperature within the range 180-260° C.
